(19) United States
(12) Patent Application Publication
(10) Pub. No.: US 2014/0343085 A1
(43) Pub. Date: Nov. 20, 2014

(54) USE OF ARYL DERIVATIVES FOR CONTROLLING ECTOPARASITES

(71) Applicant: Novartis AG, Basel (CH)

(72) Inventors: Cyril Desevaux, Saint-Aubin (CH); Martin Jung, Saint-Aubin (CH); Caroline Kablitz, Basel (CH); Steve Nanchen, Basel (CH)

(73) Assignee: Novartis AG, Basel (CH)

(21) Appl. No.: 14/361,588

(22) PCT Filed: Nov. 23, 2012
(86) PCT No.: PCT/EP2012/073475

§ 371 Date: May 29, 2014

(30) Foreign Application Priority Data
Nov. 29, 2011 (EP) 11191133.5

(51) Int. Cl. C07D 413/04; C07D 261/04

(52) U.S. Cl. CPC C07D 413/04; C07D 261/04
USPC 514/275; 514/378; 514/340

(57) ABSTRACT

The present invention relates to the use of compounds of formula (I), wherein the variables are as defined in the description, in the free form or in salt form, for the manufacture of a medicament for controlling ectoparasites on a warm-blooded animal, wherein said medicament is administered orally to the animal at a dose of from 0.1 to 100 mg/kg from 30 minutes before to 3 hours after feeding the animal with an animal food.

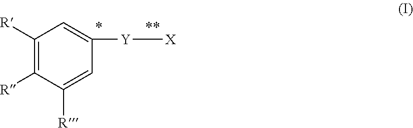

The present invention relates to the oral use of an aryl isoxazoline compound or a related heterocyclic compound in the control of parasites on warm-blooded animals.

The chemical class of aryl isoxazoline compounds has attracted a lot of attention in the agrochemical field. For example, WO2005/085216 discloses the efficacy of said class of compounds as agrochemical pest control agents.

There are also attempts to use aryl isoxazoline compounds in the veterinary field, particularly in the control of ectoparasites such as ticks and fleas. The application of the active ingredient to the animal may occur topically or parenterally. Current anti-flea and anti-tick drugs are commonly administered via spray-on or spot-on application. However, said topical applications have certain draw-backs. For example, the application of the liquid formulation to the skin or fur of a cat or dog is not very convenient and, in addition, the fur may be harmed over time; moreover, once applied the drug solution, which often has an oily consistence, will spread all over the fur or skin and remain there for a prolonged time which may cause safety and environmental issues.

Accordingly, it would be desirable to provide an oral application of the aryl isoxazoline compounds to animals. However, initial experiments of oral application to dogs and cats provided very mixed results. Following the application of an identical dose of compound in each case to various dogs or cats, tick and flea control was sometimes complete over a prolonged time and sometimes unsatisfactory. Measurements of the bioavailability confirmed a great variability of the amount of active ingredient in the blood stream.

Surprisingly, it has now been found out that the bioavailability of the aryl isoxazoline compounds in an animal body is strongly dependent on whether the medicament is applied to the animal in fed or fasted condition. In particular, it has been found that animal food strongly increases the bioavailability of aryl isoxazoline compounds in warm-blooded animals such as cats and dogs.

Accordingly, the present invention concerns the use of a compound of formula

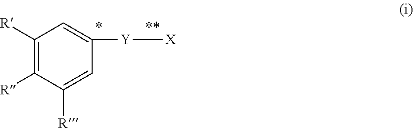

including all geometric and stereoisomers, N-oxides, S-oxides and salts thereof, wherein, R′, R″ and R′″ are each independently hydrogen, halogen, cyano, C₁-C₂-alkyl, halo-C₁-C₂-alkyl, C₁-C₂-alkoxy or C₁-C₂-haloalkoxy, subject to the proviso that at least one of R′, R″ and R′″ is not hydrogen;
*—Y—** is a radical of formula

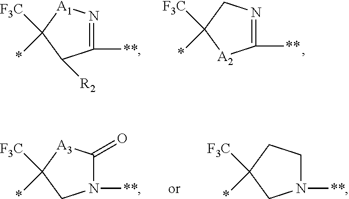

### A₁ is O, S or NR₁′, A₂ is CH₂, O or S, and A₃ is O, S or NR₁′;

R₁′ independently is as defined as R₁ below;
R₂ is H, methyl, halogen, hydroxy or methylsulfonyl;

### and X is

(a) a radical of formula

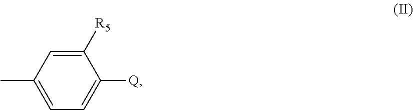

wherein R₅ is H, C₁-C₂-alkyl, C₁-C₂-haloalkyl, halogen, nitro or cyano and Q is
(i) a 5- or 6-membered heteroaromatic ring comprising 1 to 3 same or different heteroatoms selected from the group consisting of O, S and N which is further unsubstituted or substituted; or is
(ii) a group —C(O)N(R₁)-T, wherein R₁ is H, C₁-C₄-alkyl, C₂-C₄-alkylcarbonyl or C₂-C₄-alkoxycarbonyl and T is C₁-C₆-alkyl which is unsubstituted or substituted by C₃-C₆-cycloalkyl, halogen, cyano, nitro, amino, hydroxy, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, carboxy, carbamoyl, C₁-C₆-alkylcarbonylamino, C₁-C₆-haloalkylcarbonylamino, C₁-C₆-alkoxycarbonyl, sulfonamido, N-mono- or N,N, di-C₁-C₄-alkylsulfonamido, C₂-C₆-alkanoyl, unsubstituted or in the alkyl portion by halogen, cyano, ethenyl or ethynyl substituted N—C₁-C₆-alkylaminocarbonyl, or unsubstituted or halogen-, C₁-C₂-alkyl-, C₁-C₂-haloalkyl or cyano-substituted 4- to 6-membered heterocyclyl; or T is C₃-C₆-cycloalkyl or 4- to 6-membered heterocyclyl, which is each unsubstituted or substituted by halogen, C₁-C₂-alkyl, C₁-C₂-haloalkyl or cyano; or is
(iii) a radical —C(O)NH—C═N—O—C₁-C₂-alkyl, a radical —C(O)N═C—N-di-C₁-C₂-alkyl or a radical —C(O)N═C(NH₂)—O—C₁-C₂-alkyl; or is
(iv) a group —CH(R₃)—N(R₄)—C(O)-T₁, wherein R₃ is H, C₁-C₆-alkyl, C₁-C₆-haloalkyl, halogen or cyano, R₄ is H; C₁-C₄-alkyl C₂-C₄-alkylcarbonyl or C₂-C₄-alkoxycarbonyl, and T₁ is independently defined as T above;
(b) a radical of formula

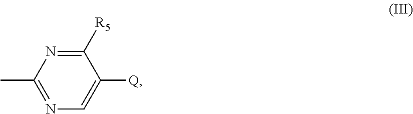

wherein R₅ is H, C₁-C₂-alkyl, C₁-C₂-haloalkyl, halogen, nitro or cyano, and Q is as defined above;
(c) a radical of formula

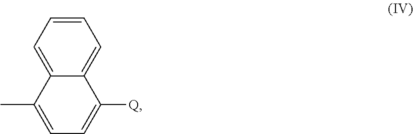

wherein Q is as defined above;
(d) a radical of formula

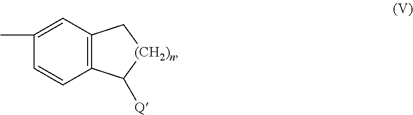

wherein n is 1 or 2 and Q′ is a group —N(R₄)—C(O)-T₂, wherein T₂ independently has the meaning of T above and R₄ is as defined above; or
(e) a radical of formula

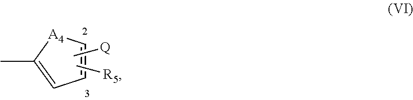

wherein A₄ is O or S and Q and R₅ are each as defined above, and wherein one of Q and R₅ is located in the 2-position and the other one in the 3-position;
for the manufacture of a medicament for controlling ectoparasites on a warm-blooded animal, wherein said medicament is administered orally to the animal at a dose of from 0.1 to 100 mg/kg from 30 minutes before to 3 hours after feeding the animal with an animal food.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers.

“Alkoxy” includes, for example, methoxy, ethoxy, n-propyloxy, isopropyloxy and the different butoxy, pentoxy and hexyloxy isomers. “Alkylthio” includes branched or straight-chain alkylthio moieties such as methylthio, ethylthio, and the different propylthio, butylthio, pentylthio and hexylthio isomers.

“Alkylsulfinyl” includes both enantiomers of an alkylsulfinyl group. Examples of “alkylsulfinyl” include CH₃S(O)—, CH₃CH₂S(O)—, CH₃CH₂CH₂S(O)—, (CH₃)₂CHS(O)— and the different butylsulfinyl, pentylsulfinyl and hexylsulfinyl isomers.

Examples of “alkylsulfonyl” include CH₃S(O)₂—, CH₃CH₂S(O)₂—, CH₃CH₂CH₂S(O)₂—, (CH₃)₂CHS(O)₂—, and the different butylsulfonyl, pentylsulfonyl and hexylsulfonyl isomers.

“N-alkylamino”, “N,N-di-alkyamino”, “N-alkylaminocarbonyl”, “N,N-di-alkyaminocarbonyl” and the like, are defined analogously to the above examples.

“Cycloalkyl” includes, for example, cyclopropyl, cyclobutyl, cyclopentyl and cyclohexyl. The term “alkylcycloalkyl” denotes alkyl substitution on a cycloalkyl moiety and includes, for example, ethylcyclopropyl, i-propylcyclobutyl, 3-methylcyclopentyl and 4-methycyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups.

The term “halogen”, either alone or in compound words such as “haloalkyl”, includes fluorine, chlorine, bromine or iodine. Further, when used in compound words such as “haloalkyl”, said alkyl may be partially or fully substituted with halogen atoms which may be the same or different. Examples of “haloalkyl” include F₃C—, ClCH₂—, CF₃CH₂— and CF₃CCl₂—. The terms “halocycloalkyl”, “haloalkoxy”, “haloalkylthio”, and the like, are defined analogously to the term “haloalkyl”. Examples of “haloalkoxy” include CF₃O—, CCl₃CH₂O—, HCF₂CH₂CH₂O— and CF₃CH₂O—. Examples of “haloalkylthio” include CCl₃S—, CF₃S—, CCl₃CH₂S— and ClCH₂CH₂CH₂S—. Examples of “haloalkylsulfinyl” include CF₃S(O)—, CCl₃S(O)—, CF₃CH₂S(O)— and CF₃CF₂S(O)—. Examples of “haloalkylsulfonyl” include CF₃S(O)₂—, CCl₃S(O)₂—, CF₃CH₂S(O)₂— and CF₃CF₂S(O)₂—.

“Alkylcarbonyl” denotes a straight-chain or branched alkyl moieties bonded to a C(═O) moiety. Examples of “alkylcarbonyl” include CH₃C(═O)—, CH₃CH₂CH₂C(═O)— and (CH₃)₂CHC(═O)—. Examples of “alkoxycarbonyl” include CH₃OC(═O)—, CH₃CH₂OC(═O), CH₃CH₂CH₂OC(═O)—, (CH₃)₂CHOC(═O)— and the different butoxy- or pentoxycarbonyl isomers, for example tert.-butoxycarbonyl (Boc).

The total number of carbon atoms in a substituent group is indicated by the “Cᵢ-Cⱼ” prefix where i and j are integers. For example, C₁-C₄ alkylsulfonyl designates methylsulfonyl through butylsulfonyl; C₂-alkoxyalkyl designates CH₃OCH₂; C₃-alkoxyalkyl designates, for example, CH₃CH(OCH₃), CH₃OCH₂CH₂ or CH₃CH₂OCH₂; and C₄-alkoxyalkyl designates the various isomers of an alkyl group substituted with an alkoxy group containing a total of four carbon atoms, examples including CH₃CH₂CH₂OCH₂ and CH₃CH₂OCH₂CH₂—.

When a compound is substituted with a substituent bearing a subscript that indicates the number of said substituents can exceed 1, said substituents (when they exceed 1) are independently selected from the group of defined substituents, e.g., (R₂)ₙ, n is 1 or 2. “Aromatic” indicates that each of the ring atoms is essentially in the same plane and has ap-orbital perpendicular to the ring plane, and in which (4n+2) π electrons, where n is a positive integer, are associated with the ring to comply with Hückel's rule.

The terms “heterocyclic ring”, “heterocycle” or “heterocyclyl” denote a ring in which at least one atom forming the ring backbone is not carbon, e.g., nitrogen, oxygen or sulfur. Typically a heterocyclic ring contains no more than 4 nitrogens, no more than 2 oxygens and no more than 2 sulfurs. Unless otherwise indicated, a heterocyclic ring can be a saturated, partially unsaturated, or fully unsaturated ring. When a fully unsaturated heterocyclic ring satisfies Hückel's rule, then said ring is also called a “heteroaromatic ring”, “aromatic heterocyclic ring”. Unless otherwise indicated, heterocyclic rings and ring systems can be attached through any available carbon or nitrogen by replacement of a hydrogen on said carbon or nitrogen.

A 4- to 6-membered nitrogen-containing heterocyclic ring may be attached to the remainder of formula (I) though any available carbon or nitrogen ring atom, unless otherwise described.

R′, R″ and R′″ are each independently of the other preferably H, halogen, CF₃ or cyano, and in particular H, Cl or F, subject to the proviso that at least one of R′, R″ and R′″ is not H. One preferred embodiment of the invention concerns compounds of formula (I), wherein R′, R″ and R′″ are each independently of the other H, chlorine or fluorine, subject to the proviso that at least one of R′, R″ and R″ is not H. One especially preferred embodiment concerns a compound of formula (I), wherein R′ and R″ are each halogen, for example chlorine or fluorine, in particular chlorine, and R″ is H, chlorine or fluorine, in particular H or chlorine and especially chlorine.

A₁ is preferably O or NH, in particular O. R₂ is preferably H or methyl, in particular H. A₂ is preferably CH₂. A₃ is preferably O or NR₁′, in particular O or NH and especially O.

One preferred embodiment of the invention relates to compounds of formula

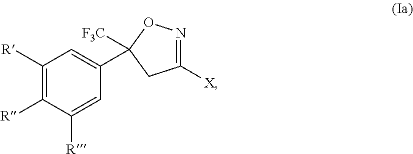

wherein for R, R′, R″ and X each the above and below given meanings and preferences apply.

A further preferred embodiment of the invention relates to compounds of formula

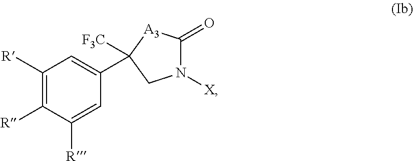

wherein for R, R′, R″ and X each the above and below given meanings and preferences apply, and A₃ is O or NH, in particular O.

A further preferred embodiment of the invention relates to compounds of formula

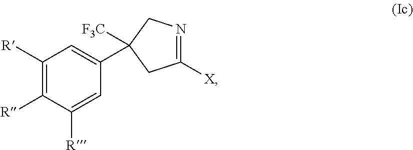

wherein for R, R′, R″ and X each the above and below given meanings and preferences apply.

Still a further preferred embodiment of the invention relates to compounds of formula

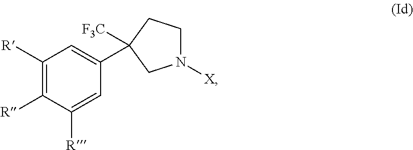

wherein for R, R′, R″ and X each the above and below given meanings and preferences apply.

In formulae (I), (Ia), (Ib), (Ic) and (Id) above, X is, for example, a radical of formula (II); according to a further embodiment, X in formulae (I), (Ia), (Ib), (Ic) and (Id) above is a radical of formula (III), (IV) or (V), more preferably a radical of formula (IV) or (V), and in particular a radical of formula (IV). According to still a further embodiment, X in formulae (I), (Ia), (Ib), (Ic) and (Id) above, is a radical of formula (VI).

The following preferences apply to the radicals of formulae (II) to (VI):

R₅ is preferably H, methyl, chlorine, nitro, cyano or CF₃, in particular H, methyl, chlorine CF₃ or cyano, in particular methyl, chlorine CF₃ or cyano, and especially methyl.

A suitable heterocyclic ring Q (embodiment (i)) is, for example, a 5- or 6-membered heteroaromatic ring having from 1 to 4, preferably from 1 to 3 same or different heteroatoms selected from the group consisting of N, O and S, which is further unsubstituted or substituted by one or more substituents selected from the group consisting of halogen, cyano, nitro, C₁-C₄-alkyl, C₁-C₄-haloalkyl, hydroxy, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, —COOH, —CONH₂, C₁-C₄-alkoxycarbonyl, sulfonamido, C₂-C₃-alkanoyl, N-mono- or N,N-di-C₁-C₄-alkylaminocarbonyl and C(S)NH₂. The heteroaromatic ring Q is preferably unsubstituted or substituted by 1 to 3, in particular 1 or 2, same or different substituents selected from the group consisting of halogen, cyano, nitro, C₁-C₂-alkyl, C₁-C₂-haloalkyl, C₁-C₂-alkoxy, C₁-C₂-haloalkoxy, C₁-C₂-haloalkylthio, C₁-C₄-alkoxycarbonyl, C₂-C₃-alkanoyl, N-mono- or N,N-di-C₁-C₃-alkylaminocarbonyl and C(S)NH₂. The heteroaromatic ring Q is most preferably unsubstituted or substituted by 1 substituent selected from the group consisting of halogen, cyano, C₁-C₂-alkoxycarbonyl, N-mono- or N,N-di-C₁-C₂-alkylaminocarbonyl and C(S)NH₂.

Examples of a 5- or 6-membered heteroaromatic rings optionally substituted with from one or more substituents include the rings Q-1 through Q-60 illustrated in Exhibit 1 wherein R is any substituent as defined before including the preferences given, and r is an integer from 0 to 4, limited by the number of available positions on each Q group. As Q-28,-Q-29, Q-35, Q-36, Q-37, Q-38, Q-39, Q-40, Q-41 and Q-42 have only one available position, for these Q groups r is limited to the integers 0 or 1, and r being 0 means that the Q group is unsubstituted and a hydrogen is present at the position indicated by (R)ᵣ.

### Exhibit 1

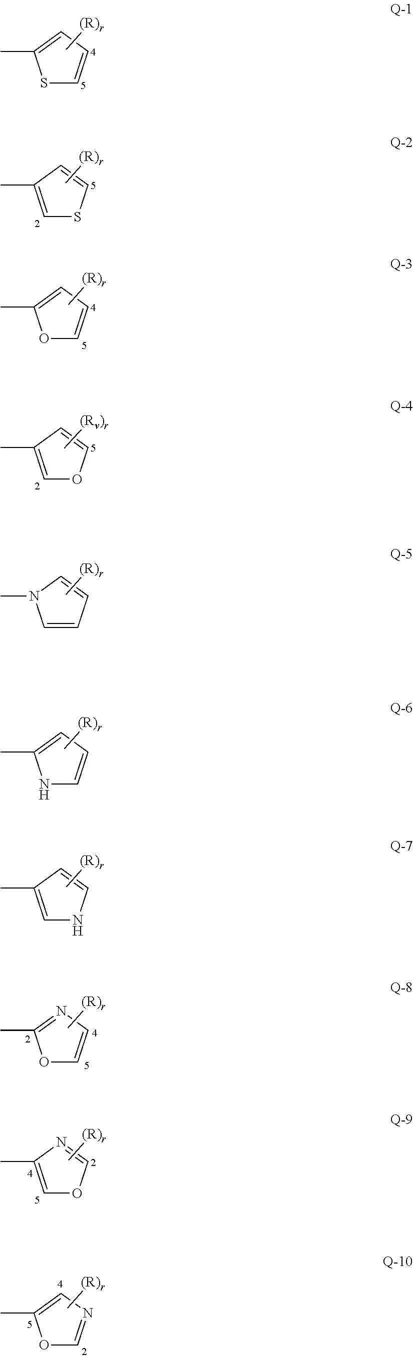

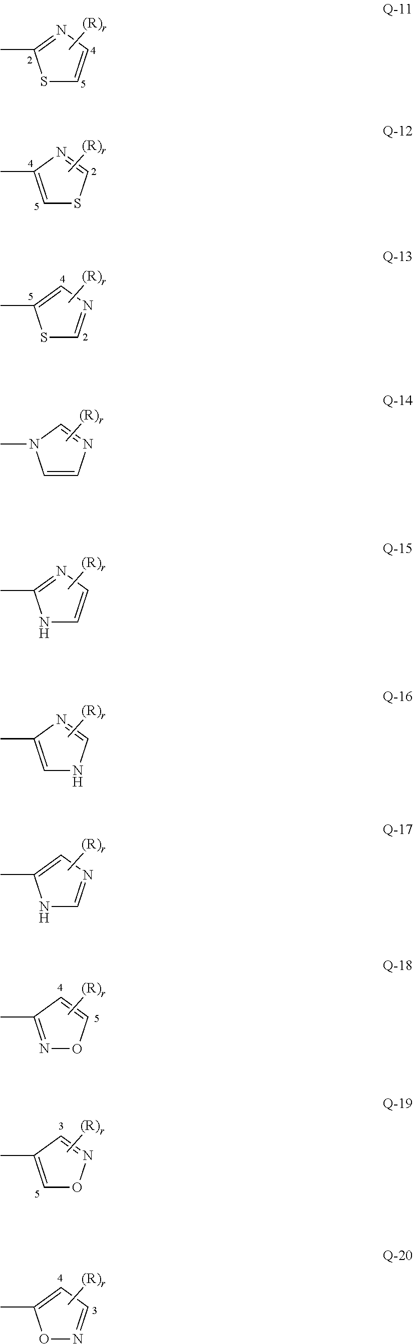

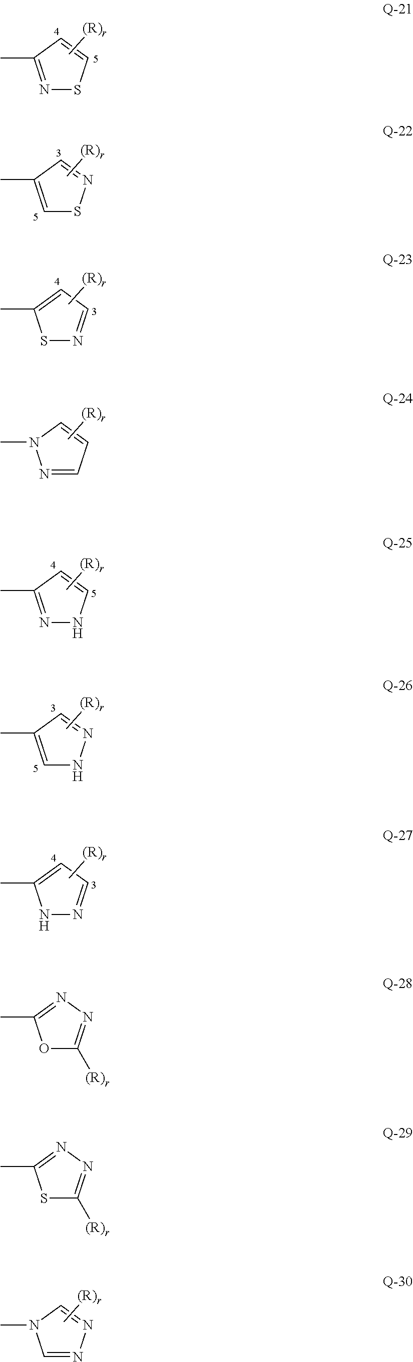

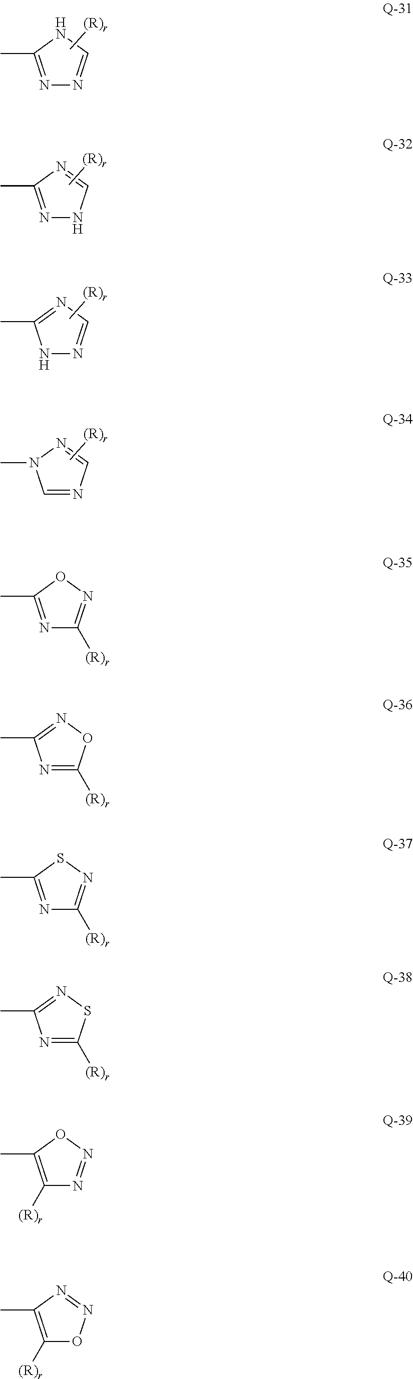

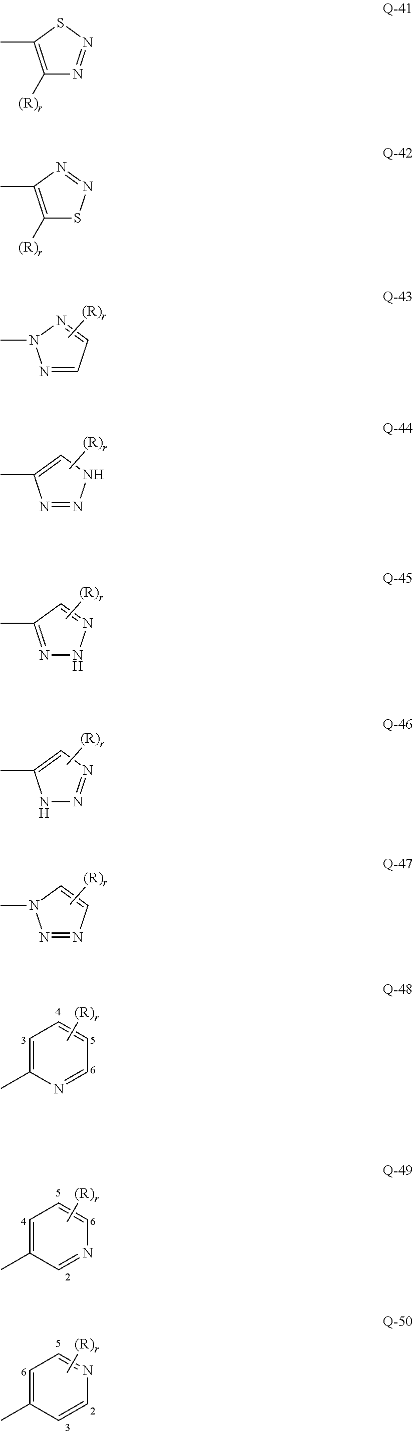

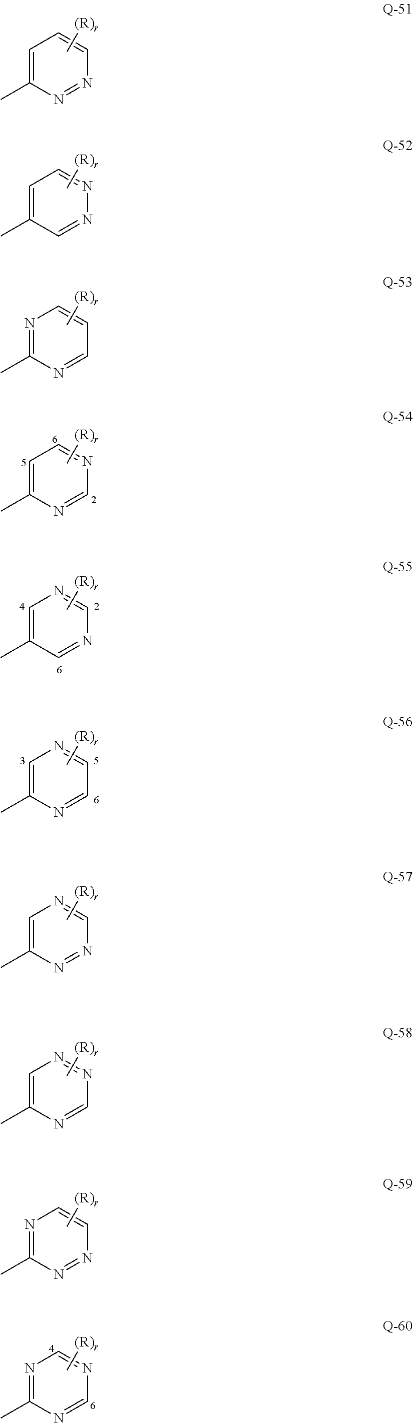

A preferred heterocyclic ring Q is of formula

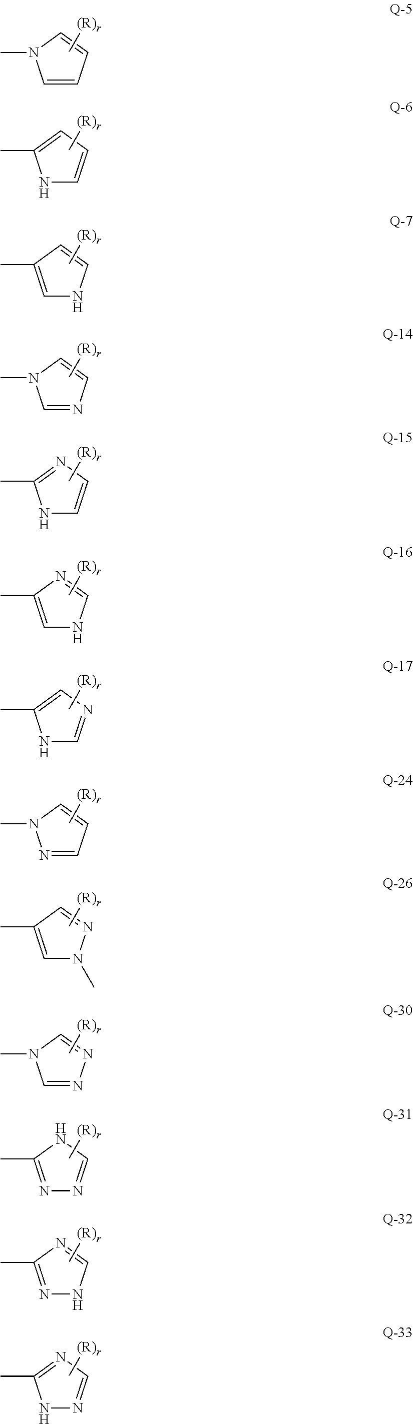

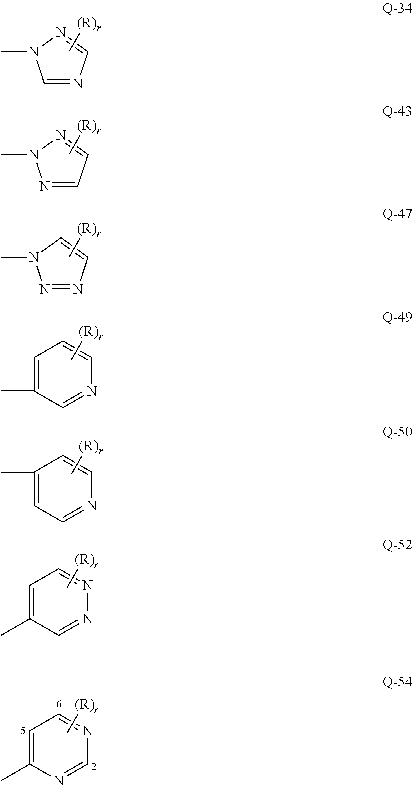

wherein r is an integer from 0 to 3 and R is independently selected from the group given before for the heteroaromatic ring including the preferences. Q is particularly preferred the unsubstituted radical Q-14, Q-24, Q-34, Q-43 or Q-47, wherein r is 0 in each case. Q is especially preferred a radical Q-14, Q-34 or Q-47, wherein r is 0.

If Q is a group —C(O)N(R₁)-T (embodiment (II)), R₁ is preferably H, methyl, ethyl or acetyl and in particular H.

T as alkyl is preferably C₁-C₄-alkyl, more preferably C₁-C₂-alkyl and particularly preferably C₁-alkyl, which is each unsubstituted or substituted as defined above.

The alkyl radical T is preferably unsubstituted or substituted by halogen; C₁-C₄-alkoxycarbonyl; N—C₁-C₆-alkylaminocarbonyl which is unsubstituted or substituted in the alkyl portion by halogen, cyano, ethenyl or ethynyl; or 5- to 6-membered heterocyclyl which is in turn unsubstituted or substituted by halogen-, C₁-C₂-alkyl- or C₁-C₂-haloalkyl.

A preferred N-alkylaminocarbonyl substituent of the alkyl radical T is N—C₁-C₂-alkylaminocarbonyl, which is unsubstituted or further substituted in the alkyl moiety by halogen, cyano, ethenyl or ethynyl. Especially preferred N-alkylaminocarbonyl substituents of the alkyl radical T are N-ethylaminocarbonyl or a radical —C(O)NH—CH₂CF₃, —C(O)NH—CH₂CN, —C(O)NH—CH₂CH═CH₂ or —C(O)NH—CH₂C≡CH.

T as N-alkylaminocarbonyl-substituted alkyl is preferably N-ethylaminocarbonylmethyl, or a radical —CH₂—C(O)NH—CH₂CF₃, —CH₂—C(O)NH—CH₂CN or —CH₂—C(O)NH—CH₂C≡CH.

If T is heterocyclyl-substituted alkyl, preferred meanings of heterocyclyl include pyridyl, pyrimidinyl, thiazolyl, oxazolyl, tetrahydrofuranyl, thietanyl or oxetanyl. Preferred heterocyclyl-substituted alkyl radicals T are in particular 2-pyridylmethyl or 2-tetrahydrofuranylmethyl.

T as heterocyclyl preferably denotes as 4- to 6-membered ring comprising 1 to 3 same or different heteroatoms selected from the group consisting of O, S and N, which is each unsubstituted or substituted by halogen, C₁-C₂-alkyl or C₁-C₂-haloalkyl.

If T is 4- to 6-membered heterocyclyl, preferred meanings of heterocyclyl include pyridyl, pyrimidyl, thiazolyl, oxazolyl, tetrahydrofuranyl, thietanyl or oxetanyl and in particular 2- 3- or 4-pyridyl, 3- 4- or 5-pyrimidyl, 2- or 3-tetrahydrofuranyl, thietan-3-yl or oxetan-3-yl and even more preferred 5-Cl-pyrimid-3-yl, 3-tetrahydrofuranyl, thietan-3-yl or oxetan-3-yl.

If Q is a group —C(O)N(R₁)-T, R₁ is preferably H, methyl, ethyl or acetyl and T is C₁-C₂-alkyl; C₁-C₂-haloalkyl; C₁-C₂-alkoxycarbonyl-C₁-C₂-alkyl; C₁-C₂-alkyl which is substituted by pyridyl, pyrimidinyl, thiazolyl, oxazolyl or tetrahydrofuranyl; C₁-C₂-alkyl which is substituted by unsubstituted or in the alkyl moiety by halogen, cyano, ethenyl or ethynyl substituted N—C₁-C₂-alkylaminocarbonyl; pyridyl; pyrimidyl; thiazolyl; oxazolyl; tetrahydrofuranyl; thietanyl; or oxetanyl.

If Q is a group —C(O)N(R₁)-T, R₁ is most preferably H, methyl or ethyl, and T is C₁-C₂-alkyl; C₁-C₂-haloalkyl; methyl which is substituted by pyridyl, pyrimidinyl, thiazolyl, oxazolyl or tetrahydrofuranyl; methyl which is substituted by N—₁-C₂-alkylaminocarbonyl or by N—C₁-C₂-alkylaminocarbonyl substituted in the alkyl moiety by halogen, cyano, ethenyl or ethynyl; pyridyl; pyrimidyl; tetrahydrofuranyl; thietanyl; or oxetanyl.

If Q is a group —C(O)N(R₁)-T, R₁ is particularly preferably H, and T is C₁-C₂-alkyl; a radical —CH₂CF₃, N-ethylaminocarbonylmethyl; a radical —CH₂—C(O)NH—CH₂CF₃, —CH₂—C(O)NH—CH₂CN or —CH₂—C(O)NH—CH₂C≡CH; 2-pyridylmethyl; 5-Cl-pyrimid-3-yl; 3-tetrahydrofuranyl; thietan-3-yl; or oxetan-3-yl.

Preferred radicals Q of embodiment (iii) are a radical —C(O)NH—C═N—O—CH₃, a radical —C(O)N═C—N-di-CH₃ or a radical —C(O)N═C(NH₂)—O—CH₃.

If Q is a group —CH(R₃)—N(R₄)—C(O)-T₁ (embodiment (iv)), R₃ is preferably H or C₁-C₂-alkyl or cyano, more preferably H or methyl, and in particular H. R₄ is preferably H or C₁-C₂-alkyl, in particular H.

R₄ is preferably H or C₁-C₂-alkyl, in particular H.

T₁ as optionally substituted alkyl is preferably straight-chain or branched C₁-C₄-alkyl, which is each unsubstituted or substituted by C₃-C₆-cycloalkyl, halogen, cyano, C₁-C₄-alkoxy, Cᵣ C₂-haloalkoxy, C₁-C₄-alkylthio, C₁-C₂-haloalkylthio, C₁-C₄-alkylsulfinyl, C₁-C₄-haloalkylsulfinyl, C₁-C₄-alkylsulfonyl, C₁-C₄-haloalkylsulfonyl, C₁-C₂-alkylcarbonylamino, C₁-C₂-haloalkylcarbonylamino or 4- to 6-membered heterocyclyl. Especially preferred alkyl radicals T₁ are straight-chain or branched C₁-C₄-alkyl or C₁-C₄-alkyl which is substituted by cyclopropyl, halogen, cyano, C₁-C₂-alkoxy, C₁-C₂-haloalkoxy, C₁-C₂-alkylthio, C₁-C₂-alkylsulfinyl, C₁-C₂-alkylsulfonyl, C₁-C₂-haloalkylcarbonylamino, pyridyl, pyrimidyl, thiazolyl, oxazolyl, thietanyl, oxetanyl, dioxolanyl, methyldioxolanyl, dioxanyl or tetrahydrofuryl.

T₁ as alkyl is especially preferred straight-chain or branched C₁-C₄-alkyl, C₁-C₃-haloalkyl, cyclopropylmethyl, cyano-C₁-C₂-alkyl, C₁-C₂-alkoxy-C₁-C₂-alkyl, C₁-C₂-alkylthio-C₁-C₂-alkyl, C₁-C₂-alkylsulfinyl-C₁-C₂-alkyl, C₁-C₂-alkylsulfonyl-C₁-C₂-alkyl, or methyl which is substituted by 1,3-dioxolan-2-yl, 2-methyl-1,3-dioxolan-2-yl or tetrahydrofuran-2- or -3-yl.

Particularly preferred alkyl radicals T₁ are straight-chain or branched C₁-C₄-alkyl; C₁-C₂-alkyl which is substituted by halogen, cyano, C₁-C₂-alkoxy, C₁-C₂-alkylthio or C₁-C₂-alkylsulfonyl; or 2-methyl-1,3-dioxolan-2-yl-methyl.

If T₁ is C₃-C₆-cycloalkyl, said cycloalkyl is preferably cyclopropyl, cyclobutyl, cyclopentyl or cyclohexyl, in particular cyclopropyl.

If T₁ is 4- to 6-membered heterocyclyl, said heterocyclyl is, for example, a 4-6-membered heteroaromatic ring, preferably a thienyl, furyl, oxazolyl, thiazolyl, pyridyl or pyrimidinyl radical, which is each unsubstituted or substituted by C₁-C₂-alkyl, C₁-C₂-haloalkyl or C₁-C₄-alkoxycarbonyl. Especially preferred heteroaromatic radicals T₁ are 2-, 3- or 4-pyridyl, 2- or 4-pyrimidinyl, 2-thiazolyl, 2-furyl or 2-thienyl.

A further preferred heterocyclic radical T₁ is, for example, a 4- to 6-membered heteroaliphatic ring selected from the group of thietanyl, for example thietan-3-yl, oxo-thietanyl, dioxo-thiethanyl, oxetanyl, for example oxetan-3-yl, azetidinyl, pyrrolidinyl, tetrahydrofuranyl, tetrahydrothiophenyl, piperidinyl, piperazinyl, morpholinyl, tetrahydropyranyl and thianyl which is each unsubstituted or substituted by C₁-C₂-alkyl, C₁-C₂-haloalkyl or C₁-C₄-alkoxycarbonyl. Especially preferred heteroaliphatic ring radicals T₁ include pyrrolidinyl, tetrahydrofuranyl, tetrahydrothiophenyl, piperidinyl, piperazinyl, morpholinyl or thianyl which are each unsubstituted or substituted by C₁-C₂-alkyl, C₁-C₂-haloalkyl or C₁-C₄-alkoxycarbonyl, and in particular pyrrolidine-1-yl, tetrahydrofuran-2-yl, tetrahydrofuran-3-yl, piperidine-1-yl, morpholine-4-yl or thiane-4-yl.

Q as a group —CH(R₃)—N(R₄)—C(O)-T₁ is most preferably a radical —CH₂—NH—C(O)—C₁-C₂-alkyl, CH₂—NH—C(O)-cyclopropyl, —CH₂—NH—C(O)—(CH₂)₁₋₂—O—C₁-C₂-alkyl, —CH₂—NH—C(O)—(CH₂)₁₋₂—S—C₁-C₂-alkyl or —CH₂—NH—C(O)—(CH₂)₁₋₂—S(O)₂—C₁-C₂-alkyl.

Particular preferred meanings of Q are a radical

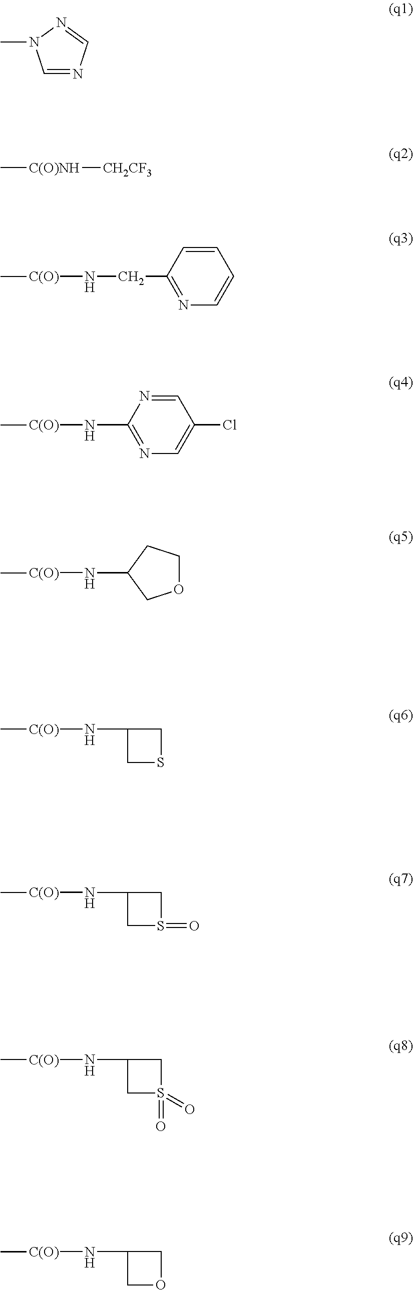

(q10) —C(O)NH—CH₂—C(O)NH—CH₂CF₃
(q11) —C(O)NH—CH₂—C(O)NH—CH₂CN
(q12) —C(O)NH—CH₂—C(O)NH—CH₂C≡CH
(q13) —C(O)NH—C═N—O—CH₃
(q14) —C(O)N═C—N(CH₃)₂
(q15) —C(O)N═C(NH₂)—O—CH₃
(q16) —CH₂—NH—C(O)—C₁-C₃-alkyl
(q17) —CH₂—NH—C(O)-cyclopropyl
(q18) —CH₂—NH—C(O)-cyclobutyl
(q19) —CH₂—NH—C(O)—C₁-C₂-haloalkyl
(q20) —CH₂—NH—C(O)—(CH₂)₁₋₂—S—C₁-C₂-alkyl
(q21) —CH₂—NH—C(O)—)—(CH₂)₁₋₂—S(O)₂—C₁-C₂-alkyl
(q22) —CH₂—NH—C(O)—(CH₂)₁₋₂—O—C₁-C₂-alkyl
(q23) .—CH₂—NH—C(O)—(CH₂)₁₋₂—CN

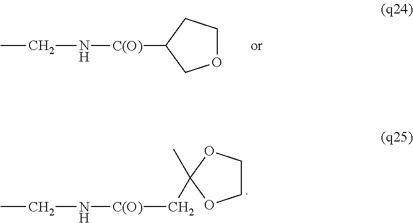

If Q′ is a group —N(R₄)—C(O)-T₂, for R₄ each the above given meanings and preferences apply independently; in addition, for T₂ each the meanings and preferences given above for T₁ apply. Particular preferred meanings of Q′ are a radical

(q26) —NH—C(O)—C₁-C₃-alkyl,
(q27) —NH—C(O)-cyclopropyl,
(q28) —NH—C(O)-cyclobutyl,
(q29) —NH—C(O)—C₁-C₂-haloalkyl,
(q30) —NH—C(O)—(CH₂)₁₋₂—S—C₁-C₂-alkyl,
(q31) —NH—C(O)—(CH₂)₁₋₂—S(O)₂—C₁-C₂-alkyl,
(q32) —NH—C(O)—(CH₂)₁₋₂—O—C₁-C₂-alkyl,
(q33) —NH—C(O)—(CH₂)₁₋₂—CN,

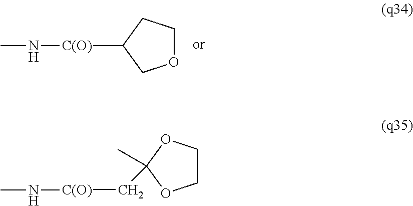

A group of preferred compounds according to the invention are those of formula

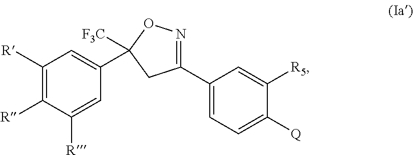

wherein R′, R″ and R″ are each independently of the other H, halogen or trifluoromethyl, subject to the proviso, that at least one of R′, R″ and R′″ is not H, R₅ is methyl, halogen, CF₃ or cyano and Q is as defined above or is preferably
(i) a radical Q-14, Q-24, Q-34, Q-43 or Q-47 mentioned above, wherein r is 0 in each case;
(ii) a radical —C(O)N(R₁)-T, wherein R₁ is H, methyl, ethyl or acetyl, and T is C₁-C₂-alkyl; C₁-C₂-haloalkyl; C₁-C₂-alkoxycarbonyl-C₁-C₂-alkyl; C₁-C₂-alkyl which is substituted by pyridyl, pyrimidinyl, thiazolyl, oxazolyl, tetrahydrofuranyl, thietanyl or oxetanyl; C₁-C₂-alkyl which is substituted by unsubstituted or in the alkyl moiety by halogen, cyano, ethenyl or ethynyl substituted N—C₁-C₂-alkylaminocarbonyl; pyridyl; pyrimidyl; thiazolyl; oxazolyl; tetrahydrofuranyl; thietanyl; or oxetanyl;
(iii) a radical —C(O)NH—C═N—O—CH₃, —C(O)N═C—N-di-CH₃ or —C(O)N═C(NH₂)—O—CH₃; or
(iv) a radical —CH(R₃)—N(R₄)—C(O)-T₁ wherein R₃ is H, C₁-C₂-alkyl or cyano, R₄ is H or C₁-C₂-alkyl, and T₁ is straight-chain or branched C₁-C₄-alkyl, C₁-C₃-haloalkyl, cyclopropylmethyl, cyano-C₁-C₂-alkyl, C₁-C₂-alkoxy-C₁-C₂-alkyl, C₁-C₂-alkylthio-C₁-C₂-alkyl, C₁-C₂-alkylsulfinyl-C₁-C₂-alkyl or C₁-C₂-alkylsulfonyl-C₁-C₂-alkyl, cyclopropyl, unsubstituted or C₁-C₂-alkyl-, C₁-C₂-haloalkyl- or C₁-C₄alkoxycarbonyl-substituted thienyl, furyl, oxazolyl, thiazolyl, pyridyl or pyrimidinyl.

A group of particularly preferred compounds according to the invention are those of formula (Ia′) above, wherein R′, R″ and R″ are each independently of the other chlorine, fluorine, or H, subject to the proviso that at least one of R′, R″ and R′″ is not H, R₅ is methyl or cyano and Q is a radical (q1) to (q25) as mentioned above.

A further group of preferred compounds according to the invention are those of formula

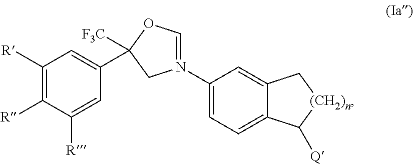

wherein R′, R″ and R″ are each independently of the other H, halogen or trifluoromethyl, subject to the proviso, that at least one of R′, R″ and R′″ is not H, n=1 or 2, and for Q′ each the above given meanings and preferences apply.

A group of particularly preferred compounds according to the invention are those of formula (Ia″) above, wherein R′, R″ and R′″ are each independently of the other chlorine, fluorine, or H, subject to the proviso that at least one of R′, R″ and R′″ is not H, n is 1, and Q is a radical (q26) to (q35) as mentioned above.

Still a further group of preferred compounds according to the invention are those of formula

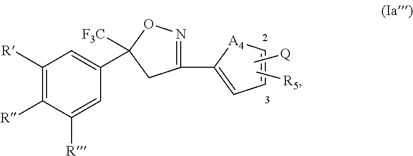

wherein R′, R″ and R″ are each independently of the other H, halogen or trifluoromethyl, subject to the proviso, that at least one of R′, R″ and R′″ is not H, R₅ is methyl, halogen, CF₃ or cyano, and for A₄, Q and R₅ independently the meanings and preferences as given above apply.

According to one embodiment of the compounds of formula (Ia′″) A₄ is S, Q is located in the 2-position, R₅ is located in the 3-position, and for Q, R′, R″, R′″ and R₅ each the above given meanings and preferences apply. According to this embodiment, R′, R″ and R′″ are independently of each other chlorine, fluorine or H, subject to the proviso that at least one of R′, R″ and R′″ is not H, R₅ is methyl, halogen, CF₃ or cyano, and Q is a radical (q1) to (q25) as mentioned above.

A particularly preferred embodiment of the invention relates to compounds of the formula (Ia′″) above wherein R′ and R′″ are each independently of the other halogen, for example chlorine or fluorine, in particular chlorine, R″ is H or halogen, preferably H or chlorine and in particular chlorine, R₅ is methyl in the 3-position, and Q is a radical (q2) to (q25) as mentioned above in the 2-position.

Specific examples of said embodiment are the compounds
5-[5-(3,5-dichloro-phenyl)-5-trifluoromethyl-4,5-dihydro-isoxazol-3-yl]-3-methyl-thiophene-2-carboxylic acid [(2,2,2-trifluoro-ethylcarbamoyl)-methyl]-amide; or
5-[5-(3,5-dichloro-phenyl)-5-trifluoromethyl-4,5-dihydro-isoxazol-3-yl]-3-methyl-thiophene-2-carboxylic acid (4-trifluoromethyl-thiazol-2-yl)-amide; or
5-[5-(3,5-dichloro-phenyl)-5-trifluoromethyl-4,5-dihydro-isoxazol-3-yl]-3-methyl-thiophene-2-carboxylic acid ethylcarbamoylmethyl-amide; or
5-[5-(3,5-dichloro-phenyl)-5-trifluoromethyl-4,5-dihydro-isoxazol-3-yl]-3-methyl-thiophene-2-carboxylic acid prop-2-ynylcarbamoylmethyl-amide; or
5-[5-(3,5-dichloro-phenyl)-5-trifluoromethyl-4,5-dihydro-isoxazol-3-yl]-3-methyl-thiophene-2-carboxylic acid [(cyanomethyl-carbamoyl)-methyl]-amide; or
5-[5-(3,5-bis-trifluoromethyl-phenyl)-5-trifluoromethyl-4,5-dihydro-isoxazol-3-yl]-3-methyl-thiophene-2-carboxylic acid [(2,2,2-trifluoro-ethylcarbamoyl)-methyl]-amide; or
5-[5-(3,4,5-trichloro-phenyl)-5-trifluoromethyl-4,5-dihydro-isoxazol-3-yl]-3-methyl-thiophene-2-carboxylic acid [(2,2,2-trifluoro-ethylcarbamoyl)-methyl]-amide; or
5-[5-(3,5-dichloro-4-fluoro-phenyl)-5-trifluoromethyl-4,5-dihydro-isoxazol-3-yl]-3-methyl-thiophene-2-carboxylic acid [(2,2,2-trifluoro-ethylcarbamoyl)-methyl]-amide.

Further embodiments of the invention relate to

(i) a compound of formula (Ia′″) wherein A₄ is O, Q is located in the 2-position, R₅′ is located in the 3-position, and for Q and R₅′ each the above given meanings and preferences apply;
(ii) a compound of formula (Ia′″) wherein A₄ is S, Q is located in the 3-position, R₅′ is located in the 2-position, and for Q and R₅′ each the above given meanings and preferences apply; or
(iii) a compound of formula (Ia′″) wherein A₄ is O, Q is located in the 3-position, R₅′ is located in the 2-position, and for Q and R₅′ each the above given meanings and preferences apply.

A further group of preferred compounds according to the invention are those of formula

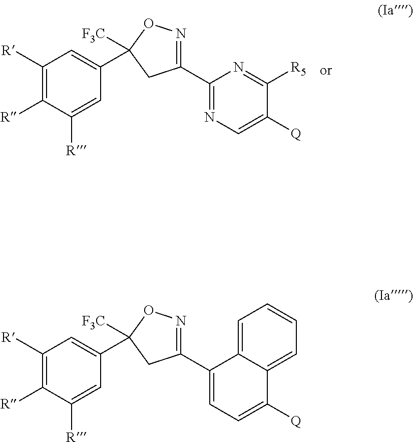

wherein R′, R″ and R″ are each independently of the other H, halogen or trifluoromethyl, subject to the proviso, that at least one of R′, R″ and R′″ is not H, R₅ is methyl, halogen, CF₃ or cyano, and for Q independently the meanings and preferences given above apply.

The compounds of formula I are known from literature, for example from, WO 2005/085216, WO 2007/026965, WO 2007/070606, WO 2007/075459, WO 2007/079162, WO 2007/108448, WO 2007/123855, WO 2008/019760, WO 2009/022746, WO 2009/035004, WO 2009/080250, WO 2009/112275 or WO2010/070068, primarily for pest control in the field of crop protection.

The compounds of formula I may be present in the form of enantiomers. The preparation and isolation of enantiomers is known per se. Accordingly, any reference to compounds of formula I hereinbefore and hereinafter is understood to include also their pure enantiomeric forms, even if the latter are not specifically mentioned in each case.

The compounds of formula (I) in general have an asymmetric C-atom at the radical *—Y—** which is of formula

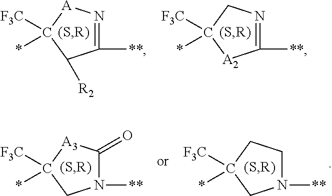

The compounds of formula (I) therefore may be employed as a racemate; in addition, a preferred embodiment of the invention concerns the use of the S-enantiomer of the compounds of formula (I) which have been found to be more active in ectoparasite control than the respective R-enantiomer in each case.

The compounds of formula I can form salts, for example acid addition salts. These are formed for example with strong inorganic acids, typically mineral acids, e.g. sulfuric acid, a phosphoric acid or a halogen acid, or with strong organic carbonic acids, typically C₁-C₄-alkanecarbonic acids substituted where appropriate for example by halogen, e.g. acetic acid, such as dicarbonic acids that are unsaturated where necessary, e.g. oxalic, malonic, maleic, fumaric or phthalic acid, typically hydroxycarbonic acids, e.g. ascorbic, lactic, malic, tartaric or citric acid, or benzoic acid, or with organic sulfonic acids, typically C₁-C₄alkane or arylsulfonic acids substituted where appropriate for example by halogen, e.g. methane-sulfonic or p-toluenesulfonic acid. In a broader sense, compounds of formula I with at least one acid group can form salts with bases. Suitable salts with bases are for example metal salts, typically alkali or alkaline earth metal salts, e.g. sodium, potassium or magnesium salts, or salts with ammonia or an organic amine, such as morpholine, piperidine, pyrrolidine, a mono-, di- or tri-lower alkylamine, e.g. ethyl, diethyl, triethyl or dimethylpropylamine, or a mono-, di- or trihydroxy-lower alkylamine, e.g. mono-, di- or triethanolamine. Furthermore, where appropriate corresponding internal salts may also be formed. The free form is preferred. Among the salts of compounds of formula I, the hydrochemically beneficial salts are preferred. Hereinbefore and hereinafter, the free compounds of formula I and their salts are understood where appropriate to include also by analogy the corresponding salts or free compounds of formula I. The same applies for the pure enantiomers of formula I and salts thereof.

A warm-blooded animal in the context of the invention is understood to include farm animals, such as cattle, horses, pigs, sheep and goats, poultry such as chickens, turkeys, guinea fowls and geese, fur-bearing animals such as mink, foxes, chinchillas, rabbits and the like, as well as companion animals such as ferrets, guinea pigs, rats, hamster, cats and dogs. A preferred warm-blooded animal according to the invention is a companion animal, in particular a cat or a dog.

In the context of the present invention, ectoparasites are understood to be in particular insects (flies, fleas, lice) or acari (mites and ticks). These include insects of the following orders: Lepidoptera, Coleoptera, Homoptera, Hemiptera, Heteroptera, Diptera, Dictyoptera, Thysanoptera, Orthoptera, Anoplura, Siphonaptera, Mallophaga, Thysanura, Isoptera, Psocoptera and Hymenoptera. However, the ectoparasites which may be mentioned in particular are those which trouble humans or animals and carry pathogens, for example flies such as Musca domestica, Musca vetustissima, Musca autumnalis, Fannia canicularis, Sarcophaga carnaria, Lucilia cuprina, Lucilia sericata, Hypoderma bovis, Hypoderma lineatum, Chrysomyia chloropyga, Dermatobia hominis, Cochliomyia hominivorax, Gasterophilus intestinalis, Oestrus ovis, biting flies such as Haematobia irritans irritans, Haematobia irritans exigua, Stomoxys calcitrans, horse-flies (Tabanids) with the subfamilies of Tabanidae such as Haematopota spp. (e.g. Haematopota pluvialis) and Tabanus spp, (e.g. Tabanus nigrovittatus) and Chrysopsinae such as Chrysops spp. (e.g. Chrysops caecutiens); Hippoboscids such as Melophagus ovinus (sheep ked); tsetse flies, such as Glossinia spp,; other biting insects like midges, such as Ceratopogonidae (biting midges), Simuliidae (Blackflies), Psychodidae (Sandflies); but also blood-sucking insects, for example mosquitoes, such as Anopheles spp, Aedes spp and Culex spp, fleas, such as Ctenocephalides felis and Ctenocephalides canis (cat and dog fleas), Xenopsylla cheopis, Pulex irritans, Ceratophyllus gallinae, Dermatophilus penetrans, blood-sucking lice (Anoplura) such as Linognathus spp, Haematopinus spp, Solenopotes spp, Pediculus humanis; but also chewing lice (Mallophaga) such as Bovicola (Damalinia) ovis, Bovicola (Damalinia) bovis and other Bovicola spp. Ectoparasites also include members of the order Acarina, such as mites (e.g. Chorioptes bovis, Cheyletiella spp., Dermanyssus gallinae, Ortnithonyssus spp., Demodex canis, Sarcoptes scabiei, Psoroptes ovis and Psorergates spp. and ticks. Known representatives of ticks are, for example, Boophilus, Amblyomma, Anocentor, Dermacentor, Haemaphysalis, Hyalomma, Ixodes, Rhipicentor, Margaropus, Rhipicephalus, Argas, Otobius and Ornithodoros and the like, which preferably infest warm-blooded animals including farm animals, poultry, fur-bearing animals, as well as companion animals such as in particular cats and dogs, but also humans.

The medicament, when administered according to the present invention is also active against all or individual development stages of animal pests showing normal sensitivity, as well as those showing resistance to widely used parasiticides. This is especially true for resistant insects and members of the order Acarina. The insecticidal, ovicidal and/or acaricidal effect of the active substances of the invention can manifest itself directly, i.e. killing the pests either immediately or after some time has elapsed, for example when moulting occurs, or by destroying their eggs, or indirectly, e.g. reducing the number of eggs laid and/or the hatching rate, good efficacy corresponding to a pesticidal rate (mortality) of at least 50 to 60% of the pests mentioned, more preferably to a mortality rate over 90%, most preferably to 95-100%.

The medicament according to the invention may contain the aryl isoxazoline alone or in combination with other biocides. The aryl oxazoline may be combined with pesticides having the same sphere of activity e.g. to increase activity, or with substances having another sphere of activity e.g. to broaden the range of activity. It can also be sensible to add so-called repellents. For example, in case of an aryl oxazoline having a particular efficacy as adulticide, i.e. since it is effective in particular against the adult stage of the target parasites, the addition of a pesticide which instead attack the juvenile stages of the parasites may be very advantageous, or vice versa. In this way, the greatest part of those parasites that produce great economic damage will be covered. Moreover, this action will contribute substantially to avoiding the formation of resistance. Many combinations may also lead to synergistic effects, i.e. the total amount of active ingredient can be reduced, which is desirable from an ecological and safety point of view. Preferred groups of combination partners and especially preferred combination partners are named in the following, whereby combinations may contain one or more of these partners in addition to the aryl oxazoline.

Suitable partners in the mixture may be biocides, e.g. the insecticides and acaricides with a varying mechanism of activity, which are named in the following and have been known to the person skilled in the art for a long time, e.g. chitin synthesis inhibitors, growth regulators; active ingredients which act as juvenile hormones; active ingredients which act as adulticides; broad-band insecticides, broad-band acaricides and nematicides; and also the well known anthelminthics and insect- and/or acarid-deterring substances, said repellents or detachers. Non-limitative examples of suitable insecticides and acaricides are mentioned in WO 2009/071500, compounds Nos. 1-284 on pages 18-21. Non-limitative examples of suitable anthelminthics are mentioned in WO 2009/071500, compounds (A1)-(A31) on page 21. Non-limitative examples of suitable repellents and detachers are mentioned in WO 2009/071500, compounds (R1)-(R3) on page 21 and 22. Non-limitative examples of suitable synergists are mentioned in WO 2009/071500, compounds (S1)-(S3) on page 22. The said partners in the mixture are best known to specialists in this field. Most are described in various editions of the Pesticide Manual, The British Crop Protection Council, London, and others in the various editions of The Merck Index, Merck & Co., Inc., Rahway, N.J., USA or in patent literature.

The aryl oxazoline may be administered in any form, for example, in liquid form such as a solution, emulsion or suspension, in semi-solid form such as a gel or paste, or in solid form such as a powder, granules, tablet, boli, capsule or chewable treat. Suitable excipients of such liquid, pasty or solid formulations are known per se, for example from WO2010070068.

According to one embodiment of the invention the aryl isoxazoline is administered in form of a tablet, capsule or granules, in particular in form of a tablet. According to a further embodiment of the invention the aryl isoxazoline is administered in form of a chewable treat. According to still a further embodiment, the aryl isoxazoline is administered in liquid or pasty form.

Application of the medicament to a warm-blooded animal, for example to a cat or dog, preferably occurs at a dose of from 0.5 to 60 mg/kg, preferably from 1 to 50 mg/kg, and in particular from 1 to 25 mg/kg of animal.

The warm-blooded animal has to be in fed condition during the application of the aryl isoxazoline. This may be achieved by feeding the animal, for example, from 3 hours before to 30 minutes after, preferably from 2 hours before to 15 minutes after, in particular from 1 hour before to concurrently with, and especially from 30 minutes before to concurrently with the administration of the medicament comprising the aryl isoxazoline. This means, the medicament is administered from 30 minutes before to 3 hours after, preferably from 15 minutes before to 2 hours after, in particular concurrently with to 1 hour after and especially concurrently with to 30 minutes after the administration of the animal food. The medicament is most conveniently administered concurrently with the administration of animal food.

Suitable animal food is known per se and may be composed of known natural and/or artificial ingredients and flavors. Typical ingredients of an animal food are one or more of the following:

(i) one or more protein sources, for example meat and/or meat byproducts, fish, vegetable protein sources or artificial protein sources, in particular meat and/or meat byproducts;
(ii) carbohydrates, for example fibers, in particular all kinds of cellulose, and non-fibers, in particular all kinds of starch, such as cereal grains, rice, wheat, corn, barley or oats;
(iii) one or more fats, for example animal fats such as lard, bacon grease, beef suet, fish fats or vegetable fats. Examples of vegetable fats are palm oil, coconut oil, cocoa fat, fats coming from olive, peanut, maize (corn oil), cottonseed, linseed, sunflower, safflower or soybean or hydrogenated vegetable oil (shortening);
(iv) minerals and vitamins;
(v) natural or artificial flavors or aromas, for example natural or artificial beef or chicken flavor, yeast or sugar.

A preferred animal food is either self-prepared or of commercial origin and contains, for example, from 30 to 100%, preferably from 50 to 100%, and in particular from 70 to 100% of the animal's daily ratio each of fat and protein, besides optionally further ingredients, for example carbohydrates, minerals, vitamins and/or flavor. Preferably, the animal food represents the warm-blooded animal's main meal of the day. The choice of animal food, whether of solid, pasty or liquid consistence, is not critical.

Preferably, the warm-blooded animal is offered from 30 to 100%, preferably from 50 to 100%, and in particular from 70 to 100% of its daily food before, concurrently with or shortly after the compound of formula (I) is administered, wherein the above-given time limits and preferences apply.

More preferably, the warm-blooded animal is offered from 30 to 100%, preferably from 50 to 100%, and in particular from 70 to 100% of its daily ratio each of fat and protein before, concurrently with or shortly after the compound of formula (I) is administered, wherein the above-given time limits and preferences apply. The animal will then automatically take up enough food in order to be in fed condition.

One embodiment of the present invention comprises administering the compound of formula (I) concurrently with 30% or more of the animal's daily food.

A further embodiment comprises administering the compound of formula (I) concurrently with or up to 1 hours after, in particular concurrently with or up to 30 minutes after the animal's main meal of the day.

The aryl isoxazoline is administered to the animal, for example once a week or less, preferably once every two weeks or less, and in particular once every four weeks or less to the animal. According to a preferred process of the invention the aryl isoxazoline is administered once every 4-6 weeks, in particular once every 4 weeks, to the target animal.

The following examples illustrate the invention without limiting it.

### EXAMPLE 1

To 12 dogs of mixed breed, the racemic compound 5-[5-(3,4,5-trichloro-phenyl)-5-trifluoromethyl-4,5-dihydro-isoxazol-3-yl]-3-methyl-thiophene-2-carboxylic acid [(2,2,2-trifluoro-ethylcarbamoyl)-methyl]-amide was administered in form of a solution at a dose of 50 mg/kg of animal, and the blood concentration of said aryl isoxazoline compound within the dogs was then monitored up to 6 days following the administration.

4 dogs (Group 1) received a full daily ration of pelleted dry food 15 to 30 minutes before the administration of the aryl isoxazoline compound; The Group 2 dogs received an identical ration of pelleted dry feed 30 minutes after the administration of the active ingredient. To the remaining 4 dogs of Group 3, the aryl isoxazoline compound was administered in fasted condition, and they received the identical ration of pelleted dry food only 5 hours following the uptake of the antiparasiticide.

Analysis of the blood concentration was performed as follows. A blood sample from the animal was subjected to a precipitation step with organic solvent and subsequently cleaned up on a 018 solid phase extraction cartridge. The eluate was evaporated to dryness and reconstituted in mobile phase and analyzed on a HPLC-MSMS using an enantioselective column (amylose-based, brand name “Chiralpak”).

Table 1 below shows the average blood concentration of the active enantiomer of the active ingredient for the Group 1, 2 and 3 dogs dependent on time.

TABLE 1

| | Average conc. of | | |
|---|---|---|---|
| | active enantiomer [ng/mL] | | |
| Time | Group 1 | Group 2 | Group 3 |
| Before treatment | <5 | <5 | <5 |
| 1 h after treatment | 1471 | 425 | 563 |
| 2 h after treatment | 2423 | 1716 | 1003 |
| 4 h after treatment | 2947 | 3540 | 1001 |
| 8 h after treatment | 3846 | 3921 | 715 |
| 1 day after treatment | 4680 | 3370 | 1379 |
| 2 days after treatment | 4256 | 2580 | 1419 |
| 3 days after treatment | 4071 | 2747 | 1150 |
| 6 days after treatment | 3746 | 2409 | 960 |

The highest blood concentration of active ingredient was obtained with the Group 1 dogs; the blood concentration of active ingredient in the Group 2 dogs allowed effective ectoparasite control as well. By contrast, the blood concentration of the active enantiomer in the Group 3 dogs never reached a level being sufficient for an effective ectoparasite control.

### EXAMPLE 2

To 4 cats, each 45 mg of the racemic compound 5-[5-(3,4,5-trichloro-phenyl)-5-trifluoromethyl-4,5-dihydro-isoxazol-3-yl]-3-methyl-thiophene-2-carboxylic acid [(2,2,2-trifluoro-ethylcarbamoyl)-methyl]-amide were administered in form of an oral solution, and the blood concentration of said aryl isoxazoline compound within the cats was then monitored according to the method as described in Example 1 up to 24 days following the administration.

2 cats (Group 1) received a full daily ration of commercial wet food 15 to 30 minutes before the administration of the aryl isoxazoline compound; The two Group 2 cats received an identical ration of wet food only 5 hours following the uptake of the aryl isoxazoline.

Table 2 below shows the average blood concentration of the active enantiomer of the active ingredient for the Group 1 and Group 2 cats dependent on time.

TABLE 2

Average conc. of active
enantiomer [ng/mL]

| Time | Group 1 | Group 2 |
|---|---|---|
| Before treatment | <10 | <10 |
| 2 h after treatment | 270 | 324 |
| 4 h after treatment | 513 | 336 |
| 8 h after treatment | 826 | 249 |
| 1 day after treatment | 1400 | 227 |
| 2 days after treatment | 1287 | 173 |
| 3 days after treatment | 1117 | 161 |
| 6 days after treatment | 1101 | 178 |
| 10 days after treatment | 745 | 126 |
| 20 days after treatment | 760 | 127 |
| 24 days after treatment | 983 | 148 |

The fed Group 1 cats developed a sufficient blood concentration of active ingredient after about 8 h and maintained it for at least 24 days. By contrast the fasted Group 2 cats never reached a level of active ingredient sufficient to provide an effective ectoparasite control.

1. A method for controlling ectoparasites on a warm-blooded animal, said method comprising orally administering to the animal a compound of formula 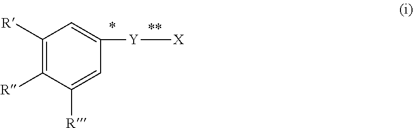
including all geometric and stereoisomers, N-oxides, S-oxides and salts thereof,
wherein, R′, R″ and R′″ are each independently hydrogen, halogen, cyano, C₁-C₂-alkyl, halo-C₁-C₂-alkyl, C₁-C₂-alkoxy or C₁-C₂-haloalkoxy, subject to the proviso that at least one of R′, R″ and R′″ is not hydrogen;
*—Y—** is a radical of formula 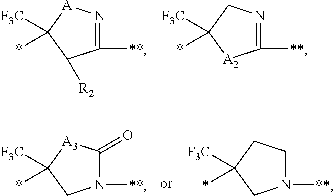
A₁ is O, S or NR₁′, A₂ is CH₂, O or S, and A₃ is O, S or NR₁′;
R₁′ independently is as defined as R₁ below;
R₂ is H, methyl, halogen, hydroxy or methylsulfonyl;
and X is
(a) a radical of formula 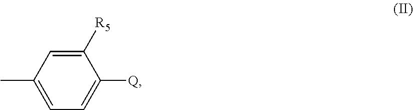
wherein R₅ is H, C₁-C₂-alkyl, C₁-C₂-haloalkyl, halogen, nitro or cyano and Q is
(i) a 5- or 6-membered heteroaromatic ring comprising 1 to 3 same or different heteroatoms selected from the group consisting of O, S and N which is further unsubstituted or substituted; or is
(ii) a group —C(O)N(R₁)-T, wherein R₁ is H, C₁-C₄-alkyl, C₂-C₄-alkylcarbonyl or C₂-C₄-alkoxycarbonyl and T is C₁-C₆-alkyl which is unsubstituted or substituted by C₃-C₆-cycloalkyl, halogen, cyano, nitro, amino, hydroxy, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₅-alkylsulfonyl, C₁-C₆-haloalkylsulfonyl, carboxy, carbamoyl, C₁-C₆-alkylcarbonylamino, C₁-C₆-haloalkylcarbonylamino, C₁-C₆-alkoxycarbonyl, sulfonamido, N-mono- or N,N, di-C₁-C₄-alkylsulfonamido, C₂-C₆-alkanoyl, unsubstituted or in the alkyl portion by halogen, cyano, ethenyl or ethynyl substituted N—C₁-C₆-alkylaminocarbonyl, or unsubstituted or halogen-, C₁-C₂-alkyl-, C₁-C₂-haloalkyl or cyano-substituted 4- to 6-membered heterocyclyl; or T is C₃-C₆-cycloalkyl or 4- to 6-membered heterocyclyl, which is each unsubstituted or substituted by halogen, C₁-C₂-alkyl, C₁-C₂-haloalkyl or cyano; or is
(iii) a radical —C(O)NH—C═N—O—C₁-C₂-alkyl, a radical —C(O)N═C—N-di-C₁-C₂-alkyl or a radical —C(O)N═C(NH₂)—O—C₁-C₂-alkyl; or is
(iv) a group —CH(R₃)—N(R₄)—C(O)-T₁, wherein R₃ is H, C₁-C₆-alkyl, C₁-C₆-haloalkyl, halogen or cyano, R₄ is H; C₁-C₄-alkyl C₂-C₄-alkylcarbonyl or C₂-C₄-alkoxycarbonyl, and T₁ is independently defined as T above;
(b) a radical of formula 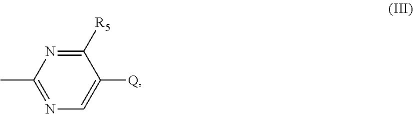
wherein R₅ is H, C₁-C₂-alkyl, C₁-C₂-haloalkyl, halogen, nitro or cyano, and Q is as defined above;
(c) a radical of formula 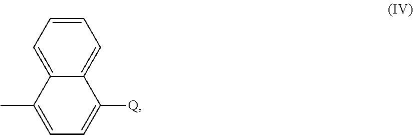
wherein Q is as defined above;
(d) a radical of formula 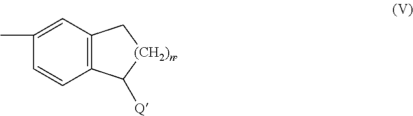
wherein n is 1 or 2 and Q′ is a group —N(R₃)—C(O)-T₂, wherein T₂ independently has the meaning of T above and R₄ is as defined above; or
(e) a radical of formula 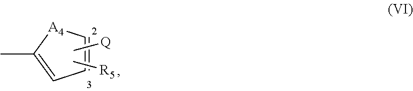
wherein A₄ is O or S and Q and R₅ are each as defined above, and wherein one of Q and R₅ is located in the 2-position and the other one in the 3-position;
wherein said compound of formula i is administered orally to the animal at a dose of from 0.1 to 100 mg/kg from 30 minutes before to 3 hours after feeding the animal with an animal food.

2. A method according to claim 1 wherein the compound of formula I comprises a compound of formula (Ia), 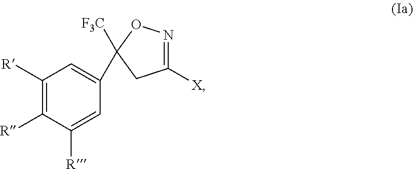
wherein R′, R″, R′″ and X are as defined.

3. A method according to claim 1, wherein R′, R″ and R′″ are each independently of the other H, halogen or trifluoromethyl, subject to the proviso, that at least one of R′, R″ and R′″ is not H.

4. A method according to claim 1 comprising a compound of formula 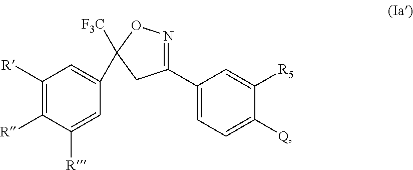
wherein R′, R″ and R′″ are each independently of the other H, halogen or trifluoromethyl, subject to the proviso, that at least one of R′, R″ and R′″ is not H, R₅ is methyl, halogen, CF₃ or cyano, and Q is as defined in claim 1.

5. A method according to claim 1 comprising a compound of formula 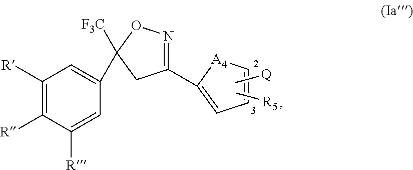
wherein R′, R″ and R′″ are each independently of the other H, halogen or trifluoromethyl, subject to the proviso, that at least one of R′, R″ and R′″ is not H, R₅′ is methyl, halogen, CF₃ or cyano, and A₄, Q and R₅ are as defined in claim 1.

6. A method according to claim 5, wherein A₄ is S, Q is located in the 2-position, R₅ is located in the 3-position.

7. A method according to claim 4, wherein R₅ is methyl, halogen, CF₃ or cyano, in particular methyl.

8. A method according to claim 1 comprising a compound of formula 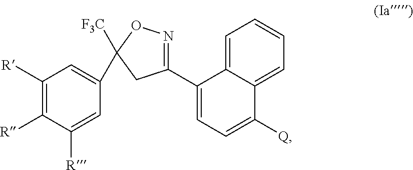
wherein R′, R″ and R′″ are each independently of the other H, halogen or trifluoromethyl, subject to the proviso, that at least one of R′, R″ and R′″ is not H, and Q is as defined in claim 1.

9. A method according to claim 4, wherein Q is
(i) a radical 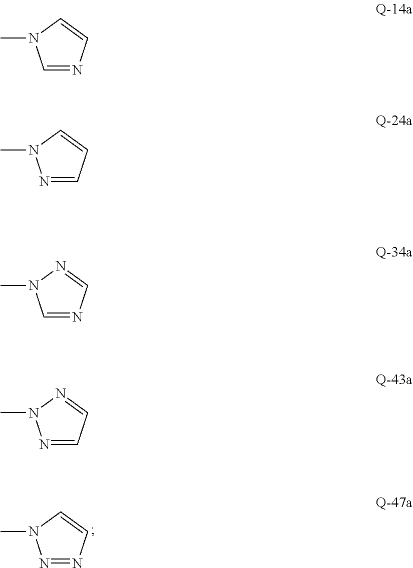
(ii) a radical —C(O)N(R₁)-T, wherein R₁ is H, methyl, ethyl or acetyl and T is C₁-C₂-alkyl; C₁-C₂-haloalkyl; C₁-C₂-alkoxycarbonyl-C₁-C₂-alkyl; C₁-C₂-alkyl which is substituted by pyridyl, pyrimidinyl, thiazolyl, oxazolyl, tetrahydrofuranyl, thietanyl or oxetanyl; C₁-C₂-alkyl which is substituted by N—C₁-C₂-alkylaminocarbonyl or by N—C₁-C₂-alkylaminocarbonyl substituted in the alkyl moiety by halogen, cyano, ethenyl or ethynyl; pyridyl; pyrimidyl; thiazolyl; oxazolyl; tetrahydrofuranyl; thietanyl; or oxetanyl;
(iii) a radical —C(O)NH—C═N—O—CH₃, —C(O)N═C—N-di-CH₃ or —C(O)N═C(NH₂)—O—CH₃; or
(iv) a group —CH(R₃)—N(R₄)—C(O)-T₁ wherein R₃ is H, C₁-C₆-alkyl, or cyano, R₄ is H; methyl, ethyl or acetyl and T₁ is straight-chain or branched C₁-C₄-alkyl or C₁-C₄-alkyl which is substituted by cyclopropyl, halogen, cyano, C₁-C₂-alkoxy, C₁-C₂-haloalkoxy, C₁-C₂-alkylthio, C₁-C₂-alkylsulfinyl, C₁-C₂-alkylsulfonyl, C₁-C₂-haloalkylcarbonylamino, pyridyl, pyrimidyl, thiazolyl, oxazolyl, thietanyl, oxetanyl, dioxolanyl, methyldioxolanyl, dioxanyl or tetrahydrofuryl.

10. A method according to claim 4, wherein Q is a radical 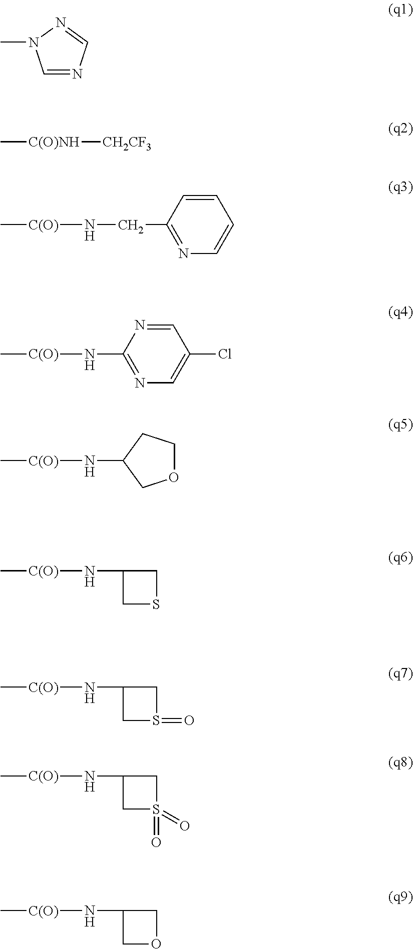
(q10) —C(O)NH—CH₂—C(O)NH—CH₂CF₃
(q11) —C(O)NH—CH₂—C(O)NH—CH₂CN
(q12) —C(O)NH—CH₂—C(O)NH—CH₂C≡CH
(q13) —C(O)NH—C═N—O—CH₃
(q14) —C(O)N═C—N(CH₃)₂
(q15) —C(O)N═C(NH₂)—O—CH₃
(q16) —CH₂—NH—C(O)—C₁-C₃-alkyl
(q17) —CH₂—NH—C(O)-cyclopropyl
(q18) —CH₂—NH—C(O)-cyclobutyl
(q19) —CH₂—NH—C(O)—C₁-C₂-haloalkyl
(q20) —CH₂—NH—C(O)—(CH₂)₁₋₂—S—C₁-C₂-alkyl
(q21) —CH₂—NH—C(O)—)—(CH₂)₁₋₂—S(O)₂—C₁-C₂-alkyl
(q22) —CH₂—NH—C(O)—(CH₂)₁₋₂—O—C₁-C₂-alkyl
(q23) .—CH₂—NH—C(O)—(CH₂)₁₋₂—CN

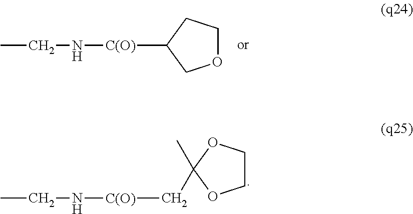

11. A method according to claim 1 comprising a compound of formula 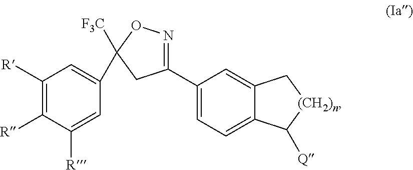
wherein R′, R″ and R′″ are each independently of the other H, halogen or trifluoromethyl, subject to the proviso, that at least one of R′, R″ and R′″ is not H, n is 1 or 2, and Q″ is as defined in claim 1.

12. A method according to claim 10, wherein n is 1 and Q″ is a radical
(q26) —NH—C(O)—C₁-C₃-alkyl,
(q27) —NH—C(O)-cyclopropyl,
(q28) —NH—C(O)-cyclobutyl,
(q29) —NH—C(O)—C₁-C₂-haloalkyl,
(q30) —NH—C(O)—(CH₂)₁₋₂—S—C₁-C₂-alkyl,
(q31) —NH—C(O)—(CH₂)₁₋₂—S(O)₂—C₁-C₂-alkyl,
(q32) —NH—C(O)—(CH₂)₁₋₂—O—C₁-C₂-alkyl,
(q33) —NH—C(O)—(CH₂)₁₋₂—CN,

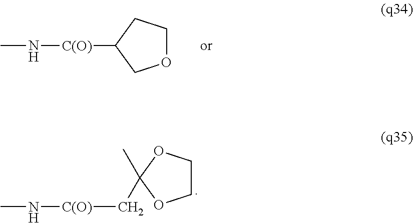

13. (canceled)

14. A method according to claim 1, wherein the aryl isoxazoline compound is administered at a dose of from 0.5 to 60 mg/kg and in particular from 1 to 25 mg/kg of animal.

15. A method according to claim 1, wherein the aryl isoxazoline compound is administered in form of a tablet or chewable treat.

16. A method according to claim 1, wherein the aryl isoxazoline compound is administered in liquid form.

17. A method according to claim 1, wherein the aryl isoxazoline compound is administered concurrently with up to 1 hour after feeding and in particular concurrently with up to 30 minutes after feeding the animal with the animal food.

18. A method according to claim 1, wherein the animal food comprises from 30 to 100%, in particular from 50 to 100%, of the animal's daily ratio of fat and protein.

19. A method according to claim 1, wherein the animal food represents the animal's main meal of the day.

20. A method according to claim 1, wherein the aryl oxazoline compound is administered once a week or less, preferably once every two weeks or less, and in particular once every four weeks or less.
